(19) United States
(12) Patent Application Publication
(10) Pub. No.: US 2022/0096020 A1
(43) Pub. Date: Mar. 31, 2022

(54) PREDICTION OF HEART FAILURE STATUS

(71) Applicant: Cardiac Pacemakers, Inc., St. Paul, MN (US)

(72) Inventors: Rezwan Ahmed, Arden Hills, MN (US); Pramodsingh Hirasingh Thakur, Woodbury, MN (US); Viktoria A. Averina, Shoreview, MN (US); Qi An, Shoreview, MN (US)

(21) Appl. No.: 17/488,962

(22) Filed: Sep. 29, 2021

Related U.S. Application Data
(60) Provisional application No. 63/085,581, filed on Sep. 30, 2020.

(51) Int. Cl. A61B 5/00; G16H 40/67; G16H 50/20

(52) U.S. Cl. CPC A61B 5/7275; A61B 5/4842; G16H 50/20; G16H 40/67

(57) ABSTRACT

Systems and methods for monitoring a heart failure (HF) patient and forecasting the patient's future HF status are discussed. A system includes a HF predictor circuit to generate a monitored sensor trend using sensor data collected up to a prediction time from the patient. The HF predictor circuit can generate a projected sensor trend for the patient over a forecast period of time in future beyond the prediction time using the monitored sensor trend of the patient and sensor trends collected from a plurality of patients. The projection can be based on a non-parametric predictor or a parametric model. A trajectory of the projected sensor trend may be displayed as a visual forecast of the patient's HF status.

### CLAIM OF PRIORITY

This application claims the benefit of priority under 35 U.S.C. § 119(e) of U.S. Provisional Patent Application Ser. No. 63/085,581, filed on Sep. 30, 2020, which is herein incorporated by reference in its entirety.

### TECHNICAL FIELD

This document relates generally to medical devices, and more particularly, to systems, devices, and methods for predicting heart failure status.

### BACKGROUND

Congestive heart failure (CHF) is a leading cause of death in the United States and globally. CHF is the loss of pumping power of the heart, and may affect left heart, right heart, or both sides of the heart, and result in the inability to deliver enough blood to meet the demands of peripheral tissues. CHF patients typically have enlarged heart with weakened cardiac muscles, resulting in reduced contractility and poor cardiac output of blood. CHF may be treated by drug therapy, or by an implantable medical device (IMD) such as for providing electrostimulation therapy. Although usually a chronic condition, CHF may occur suddenly.

Some IMDs are capable of monitoring CHF patients and detect events leading to worsening heart failure (WHF). These IMDs may include sensors to sense physiologic signals from a patient. Frequent patient monitoring may help reduce heart failure hospitalization. Identification of patient at an elevated risk of developing WHF, such as heart failure decompensation, may help ensure timely treatment and improve prognosis and patient outcome. Identifying and safely managing the patients at elevated risk of WHF may avoid unnecessary medical interventions, hospitalization, and thereby reduce healthcare cost.

### SUMMARY

This document discusses, among other things, systems, and methods for monitoring a heart failure (HF) patient and forecast the patient's future HF status. A system includes a HF predictor circuit configured to generate a monitored sensor trend using sensor data collected up to a prediction time from the patient. The HF predictor circuit can generate a projected sensor trend for the patient over a forecast period of time in future beyond the prediction time using the monitored sensor trend of the patient and information about patients' data collected from a plurality of patients. The projection can be based on a non-parametric predictor or a parametric model. A trajectory of the projected sensor trend may be displayed as a visual forecast of the patient's HF status.

Example 1 is a system for monitoring heart failure (HF) in a patient, the system comprising: a HF predictor circuit configured to: generate a monitored sensor trend using sensor data collected up to a prediction time from the patient; and generate a projected sensor trend for the patient over a forecast period of time in future beyond the prediction time using at least the monitored sensor trend of the patient, the projected sensor trend indicative of a forecast of future HF status of the patient.

In Example 2, the subject matter of Example 1 optionally includes a storage device configured to store sensor trends data collected from a plurality of patients, the stored sensor trends each including a first portion prior to a prediction time and a subsequent second portion post the prediction time, and wherein the HF predictor circuit is configured to: based on the monitored sensor trend of the patient, select at least a subset of the stored sensor trends; and generate the projected sensor trend for the patient using the monitored sensor trend and the selected subset of the stored sensor trends.

In Example 3, the subject matter of Example 2 optionally includes the monitored sensor trend that can include a composite sensor trend based on sensor data from two or more sensors associated with the patient and the stored sensor trends that can include at least one composite sensor trend based on sensor data from two or more sensors associated with one of the plurality of patients.

In Example 4, the subject matter of any one or more of Examples 2-3 optionally includes the HF predictor circuit that can be configured to: determine similarity indices between (1) the monitored sensor trend of the patient up to the prediction time and (2) respective first portions of the stored sensor trends; select at least the subset of the stored sensor trends based on the similarity indices; and generate the projected sensor trend for the patient using a central tendency of respective second portions of the selected subset of the stored sensor trends.

In Example 5, the subject matter of Example 4 optionally includes the HF predictor circuit that can be configured to determine the similarity indices using distance metrics or correlation metrics between the monitored sensor trend of the patient and the respective first portions of the stored sensor trends.

In Example 6, the subject matter of any one or more of Examples 4-5 optionally includes the stored sensor trends that can include a composite sensor trend generated using sensor data from two or more sensors associated with one of the plurality of patients, and the HF predictor circuit that can be configured to: identify, from the two or more sensors used for generating the composite sensor trend, one or more sensors with respective sensor trends that are substantially similar to the composite sensor trend; determine similarity indices between (1) the monitored sensor trend of the patient and (2) respective first portions of the sensor trends corresponding to the identified one or more sensors; select a subset of the sensor trends corresponding to the identified one or more sensors with respective similarity indices exceeding a threshold; and for the patient, generate the projected sensor trend using a central tendency of respective second portions of the selected subset of the sensor trends corresponding to the identified one or more sensors.

In Example 7, the subject matter of any one or more of Examples 2-6 optionally includes the HF predictor circuit that can be configured to: generate a parametric predictive model representing a relationship between (1) respective first portions of the selected subset of the sensor trends and (2) respective second portions of the selected subset of the sensor trends; and apply the monitored sensor trend to the parametric predictive model to generate the projected sensor trend for the patient.

In Example 8, the subject matter of Example 7 optionally includes the parametric predictive model that can be a regression model.

In Example 9, the subject matter of any one or more of Examples 2-8 optionally includes the HF predictor circuit that can be configured to: identify, from the plurality of patients with sensor trends stored in the storage device, one or more matching patients with respective demographic or medical history information substantially similar to that of the patient; and select the subset of the stored sensor trends from the one or more matching patients.

In Example 10, the subject matter of any one or more of Examples 2-9 optionally includes the HF predictor circuit that can be configured to generate, using the selected subset of the stored sensor trends, at least one of an upper bound of the projected sensor trend, a lower bound of the projected sensor trend, or a confidence interval about the projected sensor trend.

In Example 11, the subject matter of Example 10 optionally includes the HF predictor circuit that can be configured to, for the patient, generate an alert based on one or more of the projected sensor trend, the upper bound, the lower bound, or the confidence interval of the projected sensor trend.

In Example 12, the subject matter of any one or more of Examples 1-11 optionally includes the prediction time that can include at least one of: a time of detection of worsening heart failure (WHF) onset; a time of detection of WHF termination; or a periodic prediction time.

In Example 13, the subject matter of any one or more of Examples 1-12 optionally includes a display configured to display a trajectory of the projected sensor trend over the forecast period.

In Example 14, the subject matter of Example 13 optionally includes the display that can be configured to display at least one of an upper bound of the projected sensor trend, a lower bound of the projected sensor trend, or a confidence interval band about the projected sensor trend.

In Example 15, the subject matter of Example 14 optionally includes the display that can be configured to generate a visual comparison between (1) a sensor trend of measured sensor data during a time period beyond the prediction time and (2) the projected sensor trend during the time period beyond the prediction time.

Example 16 is a method for monitoring HF in a patient. The method comprises steps of: generating a monitored sensor trend using sensor data collected up to a prediction time from the patient; and generating, for the patient, a projected sensor trend over a forecast period of time in future beyond the prediction time using at least the monitored sensor trend, the projected sensor trend indicative of a forecast of future HF status of the patient.

In Example 17, the subject matter of Example 16 optionally includes receiving sensor trends data collected from a plurality of patients and stored in a storage device, the sensor trends each including a first portion prior to a prediction time and a subsequent second portion post the prediction time; selecting at least a subset of the stored sensor trends based on the monitored sensor trend of the patient; and generating the projected sensor trend for the patient further using the selected subset of the stored sensor trends.

In Example 18, the subject matter of Example 17 optionally includes the monitored sensor trend that can include a composite sensor trend based on sensor data from two or more sensors associated with the patient; and the stored sensor trends include at least one composite sensor trend based on sensor data from two or more sensors associated with one of the plurality of patients.

In Example 19, the subject matter of any one or more of Examples 17-18 optionally includes: selecting at least the subset of the stored sensor trends is based on similarity indices between (1) the monitored sensor trend of the patient up to the prediction time and (2) respective first portions of the stored sensor trends; and generating the projected sensor trend includes using a central tendency of respective second portions of the selected subset of the stored sensor trends.

In Example 20, the subject matter of any one or more of Examples 17-19 optionally includes: generating a parametric predictive model representing a relationship between (1) respective first portions of the selected subset of the sensor trends and (2) respective second portions of the selected subset of the sensor trends; and applying the monitored sensor trend to the parametric predictive model to generate the projected sensor trend for the patient.

In Example 21, the subject matter of any one or more of Examples 17-20 optionally includes selecting at least the subset of the stored sensor trends which can include: identifying, from the plurality of patients with sensor trends stored in the storage device, one or more matching patients with respective demographic or medical history information substantially similar to that of the patient; and selecting the subset of the stored sensor trends from the one or more matching patients.

In Example 22, the subject matter of any one or more of Examples 16-21 optionally includes the prediction time that can include at least one of: a time of detection of worsening heart failure (WHF) onset; a time of detection of WHF termination; or a periodic prediction time.

In Example 23, the subject matter of any one or more of Examples 16-22 optionally includes displaying a trajectory of the projected sensor trend over the forecast period.

In Example 24, the subject matter of any one or more of Examples 16-22 optionally includes initiating or adjusting a therapy if the projected sensor trend indicates a forecast of worsening of HF status.

In Example 25, the subject matter of any one or more of Examples 1-15 optionally includes a therapy delivery circuit configured to initiate or adjust a therapy if the projected sensor trend indicates a forecast of worsening of HF status.

This Summary is an overview of some of the teachings of the present application and not intended to be an exclusive or exhaustive treatment of the present subject matter. Further details about the present subject matter are found in the detailed description and appended claims. Other aspects of the invention will be apparent to persons skilled in the art upon reading and understanding the following detailed description and viewing the drawings that form a part thereof, each of which are not to be taken in a limiting sense. The scope of the present invention is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates generally an example of a patient monitor and heart failure forecast system and portions of an environment in which the system may operate.

FIG. 2 illustrates generally an example of a heart failure monitor and forecast system.

FIGS. 3A-3B illustrate generally examples of a portion of a heart failure monitor and forecast system that may query a sensor trends database for a subset of sensor trends, and use the same to predict the patient's HF status.

FIG. 4 is a diagram illustrating an example of sensor trends in a sensor trends database.

FIGS. 5A-5B are diagrams illustrating exemplary trajectories of the projected sensor trends as a visual forecast of the HF status of a patient.

FIGS. 6A-6B are diagrams examples of comparing and contrasting a projected composite sensor trend with a measured composite sensor trend during the same forecast period.

FIG. 7 illustrates generally an example of a method for monitoring HF of a patient.

FIG. 8 illustrates generally a block diagram of an example machine upon which any one or more of the techniques discussed herein may perform.

### DETAILED DESCRIPTION

Ambulatory medical devices for monitoring HF patient may include implantable medical devices (IMD), subcutaneous medical devices, wearable medical devices or other external medical devices. An ambulatory medical device may be coupled to one or more physiologic sensors to sense electrical activity and mechanical function of the heart. The ambulatory medical device may optionally deliver therapy, such as electrical stimulation pulses, to the patient to restore or improve patient cardiac function. Some of these devices may provide diagnostic features based on physiologic information collected by multiple sensors. For example, fluid accumulation in the lungs decreases the transthoracic impedance due to the lower resistivity of the fluid than air in the lungs. The fluid accumulation may also elevate ventricular filling pressure, resulting in a louder S3 heart sound. Additionally, fluid accumulation in the lungs may irritate the pulmonary system and leads to decrease in tidal volume and increase in respiratory rate.

An IMD may generate alerts when a particular health condition or a medical event, such as one indicative of worsening heart failure (WHF), is detected. The alerts may be presented to a healthcare provider to signal the patient's condition such as a deteriorated HF status. The healthcare provider may review the physiologic data recorded in the IMD, assess patient HF status, and initiate or titrate a HF therapy.

IMDs implanted in different patients may be connected to a patient management system. Monitoring and attending to a large volume of alerts may require significant amount of time and effort from the healthcare professionals, and can be costly for a healthcare facility. Additionally, a detection of a WHF event alone, absent of information about HF progression, may add challenges to HF patient management. For example, with just an indication that a WHF event has been detected by a device (such as based sensor data), a physician that manages the patient may find it hard to tell how the patient's HF status would evolve, particularly how fast or how bad the HF status might deteriorate days or weeks from the device-detected onset of WHF. Without such an outlook, HF treatment may be delayed or not timely adjusted. It may be particularly challenging if the patient has not developed any symptoms at the time of the device-detected WHF onset and beyond for a substantial period of time. In some patients, the device-detected WHF onset may precede HF symptoms by over 10 days. This may further delay the personalized HF treatment that could have slowed down the worsening of HF in some patients had it been administered at the time of device-detected WHF onset.

For at least those reasons stated above, the present inventors have recognized that there remains a considerable need of systems, devices, and methods to forecast a HF status of a patient, and using such a forecast to guide HF treatment. Disclosed herein are systems, devices, and methods for monitoring a HF patient and forecasting the patient's future HF status. A system includes a HF predictor circuit to generate a monitored sensor trend using sensor data collected up to a prediction time from the patient. The HF predictor circuit may generate a projected sensor trend for the patient over a forecast period of time in future beyond the prediction time using the monitored sensor trend of the patient and information about patients' data collected from a plurality of patients. The projection may be based on a non-parametric predictor or a parametric model. A trajectory of the projected sensor trend may be displayed as a visual forecast of the patient's HF status.

Various embodiments described herein may help improve the medical technology of device-based heart failure patient management, particularly computerized detection and prediction of a chronic disease condition such as WHF. According to some examples, a projected sensor trend indicative of a forecast of a future HF status may be generated using historical sensor trends obtained from the patient, or from other patients, such as those with similar demographics or medical history to the patient for example. The forecast may be triggered by a specific event, such as an onset of a WHF event detected by a medical device. In some examples, the forecast may be performed periodically (e.g., nightly or weekly). The projected sensor trend may be displayed on a display unit, such as represented graphically by a trajectory of N-day forecast (e.g., a 7-day forecast or a 14-day forecast). Optionally, an upper bound, a lower bound, or a confidence interval band about the projected sensor trend may be displayed. Compared to conventional HF monitoring, the projected sensor trend and the HF trajectory of the same as described in the present disclosure can provide important prognosis information of HF status, and can assist a physician in decision making with regard to preventive treatment well before the patient develops HF symptoms. The HF forecast and trajectory described herein therefore bridges a gap between a device-based early detection of WHF and early intervention to prevent further worsening of HF, and may improve patient outcome.

The HF forecast systems and methods as discussed herein may be implemented at least partially in an existing medical-device system at little to no additional cost. The early intervention and timely treatment may help avoid unnecessary medical interventions, and reduce hospitalization and healthcare costs associated with patient management. The systems, devices, and methods discussed in this document also allows for more efficient device memory usage, such as by storing projected sensor trend and the trajectory that are clinically more relevant to HF patient management and treatment. With early intervention, fewer aggressive device therapies for treating otherwise worsened HF status are needed, device battery life may be extended, and fewer unnecessary drugs and procedures may be scheduled, prescribed, or provided. HF forecast-based therapy titration (e.g., adjusting device therapy parameters) may not only improve therapy efficacy and patient outcome, but may save device power. As such, overall system cost savings may be realized.

Although the discussion in this document focuses HF forecast, this is meant only by way of example and not limitation. It is within the contemplation of the inventors, and within the scope of this document, that the systems, devices, and methods discussed herein may also be used to forecast future progression of other diseases or conditions such as cardiac arrhythmias, syncope, respiratory disease, or renal dysfunctions, among other medical conditions. Additionally, although systems and methods are described as being operated or exercised by clinicians, the entire discussion herein applies equally to organizations, including hospitals, clinics, and laboratories, and other individuals or interests, such as researchers, scientists, universities, and governmental agencies, seeking access to the patient data.

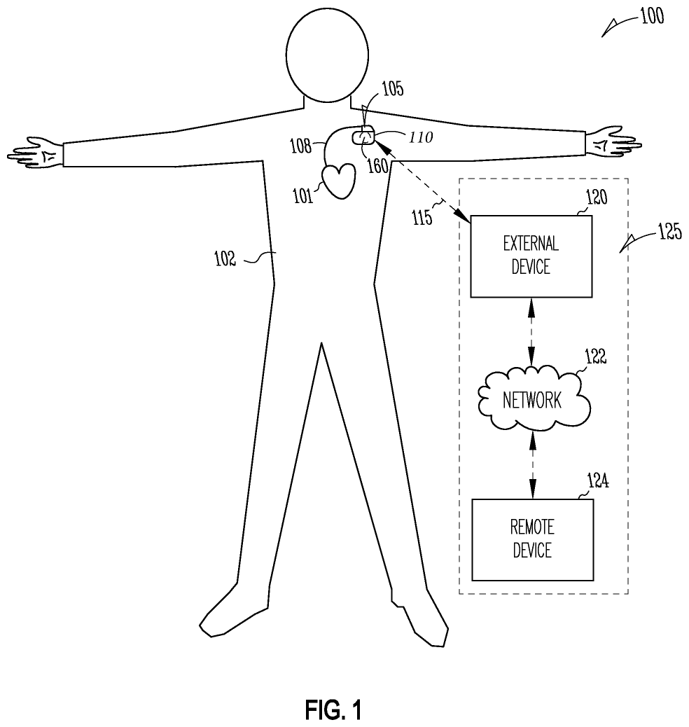

FIG. 1 illustrates generally an example of a patient monitor and heart failure forecast system 100 and portions of an environment in which the system 100 may operate. The patient monitor and heart failure forecast system 100 may chronically monitor a patient 102 to assess patient risk of developing WHF. Portions of the system 100 may be ambulatory. Portions of the system 100 may be disposed in a patient home or office, a hospital, a clinic, or a physician's office.

As illustrated in FIG. 1, the patient monitor and heart failure forecast system 100 may include an ambulatory system 105 associated with the patient 102, an external system 125, and a telemetry link 115 providing for communication between the ambulatory system 105 and the external system 125. The ambulatory system 105 may include an ambulatory medical device (AMD) 110. In an example, the AMD 110 may be an implantable device subcutaneously implanted in a chest, abdomen, or other parts of the patient 102. Examples of the implantable device may include, but are not limited to, pacemakers, pacemaker/defibrillators, cardiac resynchronization therapy (CRT) devices, cardiac remodeling control therapy (RCT) devices, neuromodulators, drug delivery devices, biological therapy devices, diagnostic devices such as cardiac monitors or loop recorders, or patient monitors, among others. The AMD 110 may include a subcutaneous medical device such as a subcutaneous monitor or diagnostic device, external monitoring or therapeutic medical devices such as automatic external defibrillators (AEDs) or Holter monitors, or wearable medical devices such as patch-based devices, smart wearables, or smart accessories.

By way of example and not limitation, the AMD 110 may be coupled to a lead system 108. The lead system 108 may include one or more transvenously, subcutaneously, or non-invasively placed leads or catheters. Each lead or catheter may include one or more electrodes. The arrangements and uses of the lead system 108 and the associated electrodes may be determined using the patient need and the capability of the AMD 110. The associated electrodes on the lead system 108 may be positioned at the patient's thorax or abdomen to sense a physiologic signal indicative of cardiac activity, or physiologic responses to diagnostic or therapeutic stimulations to a target tissue. By way of example and not limitation, and as illustrated in FIG. 1, the lead system 108 may be surgically inserted into, or positioned on the surface of, a heart 101. The electrodes on the lead system 108 may be positioned on a portion of a heart 101, such as a right atrium (RA), a right ventricle (RV), a left atrium (LA), or a left ventricle (LV), or any tissue between or near the heart portions. In some examples, the lead system 108 and the associated electrodes may alternatively be positioned on other parts of the body to sense a physiologic signal containing information about patient heart rate or pulse rate. In an example, the ambulatory system 105 may include one or more leadless sensors not being tethered to the AMD 110 via the lead system 108. The leadless ambulatory sensors may be configured to sense a physiologic signal and wirelessly communicate with the AMD 110.

The AMD 110 may include a hermetically sealed can that houses one or more of a sensing circuit, a control circuit, a communication circuit, and a battery, among other components. The sensing circuit may sense a physiologic signal, such as by using a physiologic sensor or the electrodes associated with the lead system 108. The physiologic signals may contain information about patient physiologic response to a precipitating event associated with onset of a future WHF event. The physiologic signal may represent changes in patient hemodynamic status. Examples of the physiologic signal may include one or more of electrocardiogram, intracardiac electrogram, arrhythmia, heart rate, heart rate variability, intrathoracic impedance, intracardiac impedance, arterial pressure, pulmonary artery pressure, left atrial pressure, right ventricular (RV) pressure, left ventricular (LV) coronary pressure, coronary blood temperature, blood oxygen saturation, one or more heart sounds, intracardiac acceleration, physical activity or exertion level, physiologic response to activity, posture, respiratory rate, tidal volume, respiratory sounds, body weight, or body temperature.

The AMD 110 may include a heart failure predictor circuit 160 configured to forecast the patient's future HF status. The heart failure predictor circuit 160 may receive sensor data collected from a patient up to a specific prediction time, such as an onset of WHF as detected by the AMD 110, and generate a monitored sensor trend using the received sensor data. The heart failure predictor circuit 160 may further receive information about patients' data collected from a plurality of patients, such as sensor trends generated using the patients' historical sensor data. The patients' data may be stored in a storage device. The heart failure predictor circuit 160 may generate a projected sensor trend for the patient over a forecast period of time in future beyond the prediction time (e.g., the device-detected WHF onset) using the monitored sensor trend of the patient and the information about patients' data. The projected sensor trend is indicative of a forecast of the patient's future HF status. In an example, a predictive model may be established using the stored patients' sensor trends. The predictive model may be a non-parametric model or a parametric model, and represents a relationship between predictor variables (such as respective first portions of the stored sensor trends prior to respective prediction times) and response variables (such as respective second portions of the stored sensor trend post the respective prediction times). The heart failure predictor circuit 160 may apply the monitored sensor trend to the predictive model to generate the projected sensor trend. The heart failure predictor circuit 160 may optionally generate an upper bound, a lower bound, or a confidence interval about the projected sensor trend. In some examples, the heart failure predictor circuit 160 may generate, for the patient, different levels of alert based on the projected sensor trend, or optionally based on one or more of the upper bound, the lower bound, or the confidence interval about the projected sensor trend.

The AMD 110 may include a therapy unit that may generate and deliver a therapy to the patient. The therapy may be preventive (e.g., to prevent development into a full-blown WHF event), or therapeutic (e.g., to treat heart failure or alleviate complications) in nature, and may modify, restore, or improve patient physiologic functionalities. Examples of the therapy may include electrical, magnetic, or other forms of therapy. In some examples, the AMD 110 may include a drug delivery system such as a drug infusion pump device to deliver drug therapy to the patient. In some examples, the AMD 110 may monitor patient physiologic responses to the delivered to assess the efficacy of the therapy.

The external system 125 may include a dedicated hardware/software system such as a programmer, a remote server-based patient management system, or alternatively a system defined predominantly by software running on a standard personal computer. The external system 125 may manage the patient 102 through the AMD 110 connected to the external system 125 via a communication link 115. This may include, for example, programming the AMD 110 to perform one or more of acquiring physiologic data, performing at least one self-diagnostic test (such as for a device operational status), analyzing the physiologic data to generate a projected sensor trend, or optionally delivering or adjusting a therapy to the patient 102. The external system 125 may communicate with the AMD 110 via the communication link 115. The device data received by the external system 125 may include real-time or stored physiologic data from the patient 102, diagnostic data, responses to therapies delivered to the patient 102, or device operational status of the AMD 110 (e.g., battery status and lead impedance). The communication link 115 may be an inductive telemetry link, a capacitive telemetry link, or a radio-frequency (RF) telemetry link, or wireless telemetry based on, for example, “strong” Bluetooth or WEE 802.11 wireless fidelity “WiFi” interfacing standards. Other configurations and combinations of patient data source interfacing are possible.

By way of example and not limitation, the external system 125 may include an external device 120 in proximity of the AMD 110, and a remote device 124 in a location relatively distant from the AMD 110 in communication with the external device 120 via a telecommunication network 122. Examples of the external device 120 may include a programmer device. The network 122 may provide wired or wireless interconnectivity. In an example, the network 122 may be based on the Transmission Control Protocol/Internet Protocol (TCP/IP) network communication specification, although other types or combinations of networking implementations are possible. Similarly, other network topologies and arrangements are possible.

The remote device 124 may include a centralized server acting as a central hub for collected patient data storage and analysis. The patient data may include data collected by the AMD 110, and other data acquisition sensors or devices associated with the patient 102. The server may be configured as a uni-, multi- or distributed computing and processing system. In an example, the remote device 124 may include a data processor configured to perform heart failure detection or risk stratification using respiration data received by the AMD 110. Computationally intensive algorithms, such as machine-learning algorithms, may be implemented in the remote device 124 to process the data retrospectively to detect WHF or analyze patient WHF risk. The remote device 124 may generate an alert notification. The alert notifications may include a Web page update, phone or pager call, E-mail, SMS, text or “Instant” message, as well as a message to the patient and a simultaneous direct notification to emergency services and to the clinician. Other alert notifications are possible.

One or more of the external device 120 or the remote device 124 may output the WHF detection or the forecast of future HF status to a system user such as the patient or a clinician. The external device 120 or the remote device 124 may include respective display for displaying a trajectory of the projected sensor trend over the time period beyond the specific prediction time. In some examples, one or more of the upper bound, the lower bound, or the confidence interval about the projected sensor trend may be graphically or textually displayed along with the trajectory of the projected sensor trend. Other information may also be displayed, such as the physiologic data acquired by the AMD 110, patient information including patient demographics, medical history such as medication, treatment, or readmission information, among others. The information may be presented in a table, a chart, a diagram, or any other types of textual, tabular, or graphical presentation formats. The external device 120 or the remote device 124 may include a printer for printing hard copies of signals and information related to the generation of the projected sensor trend. The presentation of the output information may include audio or other media format. In an example, the output unit 254 may generate alerts, alarms, emergency calls, or other forms of warnings to signal the system user about the WHF detection or the HF status forecast. The clinician may review, perform further analysis, or adjudicate the WHF detection or the forecast of future HF status. The WHF detection or the HF status forecast, optionally along with the data acquired by the AMD 110 and other data acquisition sensors or devices, may be output to a process such as an instance of a computer program executable in a microprocessor. In an example, the process may include an automated generation of recommendations for initiating or adjusting a therapy, or a recommendation for further diagnostic test or treatment.

Portions of the AMD 110 or the external system 125 may be implemented using hardware, software, firmware, or combinations thereof. Portions of the AMD 110 or the external system 125 may be implemented using an application-specific circuit that may be constructed or configured to perform one or more particular functions, or may be implemented using a general-purpose circuit that may be programmed or otherwise configured to perform one or more particular functions. Such a general-purpose circuit may include a microprocessor or a portion thereof, a microcontroller or a portion thereof, or a programmable logic circuit, a memory circuit, a network interface, and various components for interconnecting these components. For example, a “comparator” may include, among other things, an electronic circuit comparator that may be constructed to perform the specific function of a comparison between two signals or the comparator may be implemented as a portion of a general-purpose circuit that may be driven by a code instructing a portion of the general-purpose circuit to perform a comparison between the two signals.

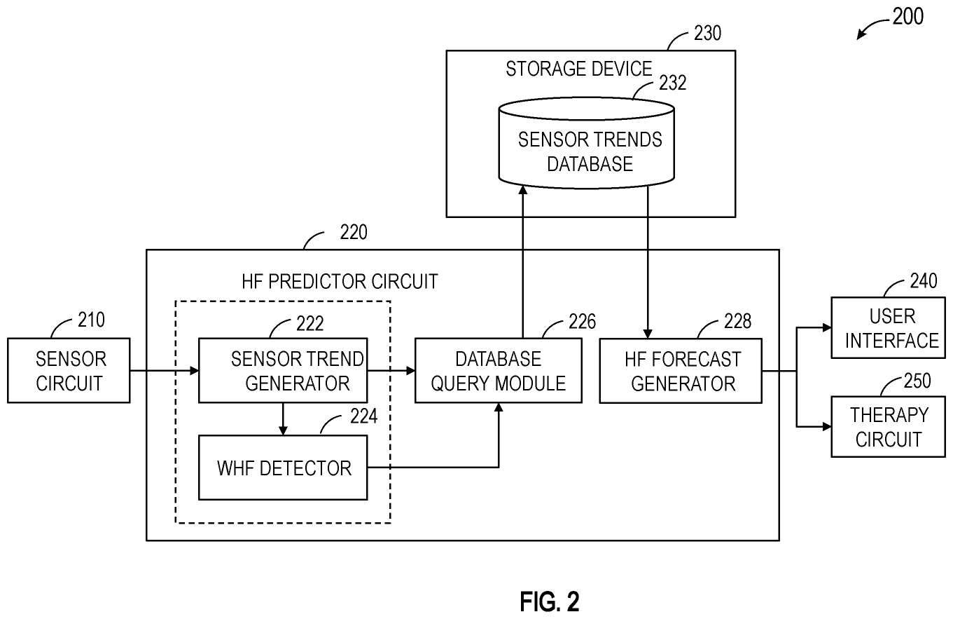

FIG. 2 illustrates generally an example of a heart failure monitor and forecast system 200 that may be configured to detect a WHF event from a patient and forecast the patient's future HF status. At least a portion of the heart failure monitor and forecast system 200 may be implemented in the AMD 110, the external system 125 such as one or more of the external device 120 or the remote device 124, or distributed between the AMD 110 and the external system 125. In the example as illustrated in FIG. 2, the heart failure monitor and forecast system 200 may include one or more of a sensor circuit 210, a heart failure predictor circuit 220, a storage device 230, a user interface 240, and an optional therapy circuit 250 for delivering a heart failure therapy.

The sensor circuit 210 may include a sense amplifier circuit to sense at least one physiologic signal from a patient. The sensor circuit 210 may be coupled to an implantable, wearable, or otherwise ambulatory sensor or electrodes associated with the patient. The sensor may be incorporated into, or otherwise associated with an ambulatory device such as the AMD 110. Examples of the physiologic signals for detecting the precipitating event may include surface electrocardiography (ECG) sensed from electrodes placed on the body surface, subcutaneous ECG sensed from electrodes placed under the skin, intracardiac electrogram (EGM) sensed from the one or more electrodes on the lead system 108, heart rate signal, physical activity signal, or posture signal, a thoracic or cardiac impedance signal, arterial pressure signal, pulmonary artery pressure signal, left atrial pressure signal, RV pressure signal, LV coronary pressure signal, coronary blood temperature signal, blood oxygen saturation signal, heart sound signal, physiologic response to activity, apnea hypopnea index, one or more respiration signals such as a respiratory rate signal or a tidal volume signal, brain natriuretic peptide, blood panel, sodium and potassium levels, glucose level and other biomarkers and bio-chemical markers, among others. In some examples, the physiologic signals sensed from a patient may be stored in a storage device, such as an electronic medical record system, and the sensor circuit 210 may be configured to receive a physiologic signal from the storage device in response to a user input or triggered by a specific event. The sensor circuit 210 may include one or more sub-circuits to digitize, filter, or perform other signal conditioning operations on the sensed physiologic signal.

The heart failure predictor circuit 220 may be configured to forecast the patient's future HF status using the sensed physiologic signal. The heart failure predictor circuit 220 may be implemented as a part of a microprocessor circuit, which may be a dedicated processor such as a digital signal processor, application specific integrated circuit (ASIC), microprocessor, or other type of processor for processing information including physical activity information. Alternatively, the microprocessor circuit may be a general-purpose processor that may receive and execute a set of instructions of performing the functions, methods, or techniques described herein.

The heart failure predictor circuit 220 may include circuit sets comprising one or more other circuits or sub-circuits including a sensor trend generator 222, a worsening heart failure (WHF) detector 224, a database query module 226, and a heart failure forecast generator 228. These circuits or sub-circuits may, either individually or in combination, perform the functions, methods or techniques described herein. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

The sensor trend generator 222 may generate a sensor trend using the sensor data collected by the sensor circuit 210 up to a specific prediction time. Such a sensor trend is hereinafter referred to as a monitored sensor trend (to distinguish from historical sensor trends stored in the storage device 230, as to be discussed below). In an example, the monitored sensor trend may be a time-series of sensor measurements (e.g., signal amplitudes at different time instants). In another example, the monitored sensor trend may be a time-series of sensor signal metric values. A sensor signal metric may include a statistical or morphological feature extracted from the sensor signal. By way of example and not limitation, the signal metrics may include heart rate, heart rate variability, cardiac activation timings, morphological features from the ECG or EGM, thoracic or cardiac impedance magnitude within a specified frequency range, intensities or timings of S1, S2, S3, or S4 heart sounds, systolic blood pressure, diastolic blood pressure, mean arterial pressure, or timing of a pressure metric with respect to a fiducial point, among others. The sensor signal metric may be trended over time to produce a monitored sensor trend.

In some examples, the monitored sensor trend may be a composite sensor trend based on a fusion of sensor data collected from two or more different sensors associated with the patient, a fusion of two or more different sensor metrics derived from the same sensor, or a fusion of two or more different sensor metrics respectively derived from different sensors associated with the patient. Various fusion algorithms may be used to compute a composite metric. In an example, the composite metric may be generated using a weighted combination of sensor data or a weighted combination of sensor signal metrics. The composite sensor trend may then be generated by trending the composite metric over time, such as for a specific number of days (e.g., 14 days) prior and up to a specific prediction time.

The prediction time is the time when a HF status forecast is initiated. The HF status forecast may be triggered by a specific event, hereinafter referred to a forecast trigger event. Various forecast trigger events have been contemplated by the present inventors. By way of example and not limitation, the forecast trigger event may include heart failure events detected by the WHF detector 224. An example of the forecast trigger event is a WHF onset. The WHF detector 224 may detect the WHF onset in response to the monitored sensor trend exceeding an onset threshold, or falling with a specific range. Another example of the forecast trigger event is a WHF termination event following a WHF onset. The WHF detector 224 may detect the WHF termination in response to the monitored sensor trend falling below a reset threshold, or falling with a specific range.

In some examples, the WHF detector circuit 224 may detect a WHF onset or a WHF termination based on changes of the monitored sensor trend over time. In an example, the WHF detector circuit 224 may calculate a difference between short-term values and baseline values of the monitored sensor trend, and detect WHF onset or WHF termination based on such a difference. The short-term values may include statistical values such as a central tendency of the measurements of the sensor data (or the senor signal metric or the composite signal metric) within a short-term window of a first plurality of days. The baseline values may include statistical values such as a central tendency of the measurements of the sensor data (or the senor signal metric or the composite signal metric) within a long-term window of a second plurality of days preceding the short-term window in time. In some examples, a linear or nonlinear combination of the relative differences between multiple short-term values corresponding to multiple first time windows and multiple baseline values corresponding to multiple second time windows may be computed using the monitored sensor trend. The differences may be scaled by respective weight factors which may be based on timing information associated with corresponding multiple short-term window, such as described by Thakur et al., in U.S. Patent Publication 2017/0095160, entitled “PREDICTIONS OF WORSENING HEART FAILURE”, which is herein incorporated by reference in its entirety. The WHF detector 224 may detect the WHF onset in response to the scaled combination of the differences between short-term values and the baseline values exceeding an onset threshold, or falling with a specific range. Similarly, the WHF detector 224 may detect the WHF termination after an initial detection of WHF onset and in response to the scaled combination of the differences between short-term values and the baseline values falling below a reset threshold, or falling with a specific range.

Additionally or alternatively, the heart failure forecast may be triggered by other heart failure trigger events, such as admission to a hospital, discharge from a hospital, development of particular heart failure symptom or comorbidity, among other events. In some examples, the heart failure forecast may be initiated by a user command, or that the prediction time may be specified by a user, or according to a specific schedule. In some examples, the HF status forecast may be performed periodically. By way of example and not limitation, the forecast may be performed daily, nightly, weekly, biweekly, or monthly.

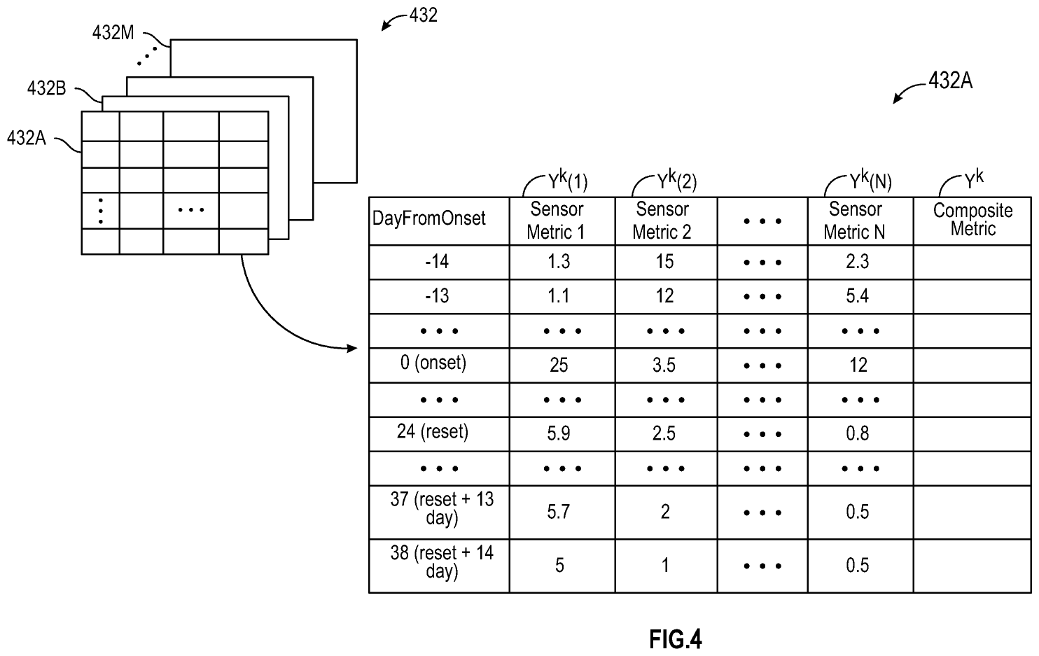

The storage device 230 can be a part of the external system 125, such as residing in the remote device 124. In some examples, the storage device 230 may be a part of a cloud-computing device or networked devices configured to provide cloud-based services such as database management. As illustrated in FIG. 2, a sensor trends database 232 may be stored and maintained in the storage device 230. The sensor trends database 232 includes sensor trends generated using sensor data collected from a plurality of patients. Similar to the monitored sensor trend for the patient, which is generated by the sensor trend generator 222, the stored sensor trends in the database 232 may include trends of sensor data, trends of sensor signal metrics, or composite sensor trends each representing a trend of a composite signal metric as a combination of two or more sensor signal metrics. Referring to FIG. 4, a sensor metric trends database 432, which is an example of the sensor trends database 232, includes a plurality of sensor metric trends 432A, 432B, . . . , 432M, each corresponding to a WHF alert indicative of a device-detected WHF onset in a patient. Each sensor metric trend (e.g., 432A) may include values of a plurality of sensor metrics (e.g., Yₖ(1), Yₖ(2), . . . , Yₖ(N)) at different times relative to the WHF onset. In an example, two or more of the N sensor metrics Yₖ(1), Yₖ(2), . . . , Yₖ(N) included in the sensor metric trends 432A may be derived from the same sensor. In another example, two or more of the N sensor metrics Yₖ(1), Yₖ(2), . . . , Yₖ(N) may be derived from different sensors. The values of the sensor metrics may be organized in a searchable data structure, such as a lookup table as illustrated in FIG. 4.

In an example, daily values of the plurality of sensor metrics may be determined, trended, and stored in the sensor trends database 232. A daily value of a sensor metric (e.g., a daily S3 heart sound intensity) may be determined using a daily average, or other central tendency, of multiple measurements of that sensor metric at different times during the day. The sensor metric values may be trended over a specific number of days with respect to the WHF onset event. The date of the device-detected WHF onset is referred to as day 0. In an example as illustrated in FIG. 4, the sensor metric trend 432A includes respective values of the N sensor metrics Yₖ(1), Yₖ(2), . . . , Yₖ(N) each trended over a trending period that extends from 14 days prior to the WHF onset (day −14) to 14 days after the date of WHF termination. The WHF termination was detected 24 days after the WHF onset (day 24). As such, the sensor metric trend 432A ends 38 days after the WHF onset (day 38).

In addition to values of individual sensor metrics, the sensor metric trend (e.g., 432A) may further include values of a composite sensor metric (e.g., Yₖ), which may be computed based on a weighted combination of the values of at least a portion of the individual sensor metrics. The composite sensor metric Yₖ may be trended over the same trending period, such as from day −14 to day 38 in an example as shown in FIG. 4.

Referring back to FIG. 2, the database query module 226 may query the sensor trends database 232 for a subset of the sensor trends that satisfy specific search criteria. The search criteria may be based on the monitored sensor trend (X) of the patient, such as produced by the sensor trend generator 222. In some examples, the search criteria may further include information about information about demographics or medical history of the patient. The stored sensor trends that satisfy the search criteria are identified as matching sensor trends.

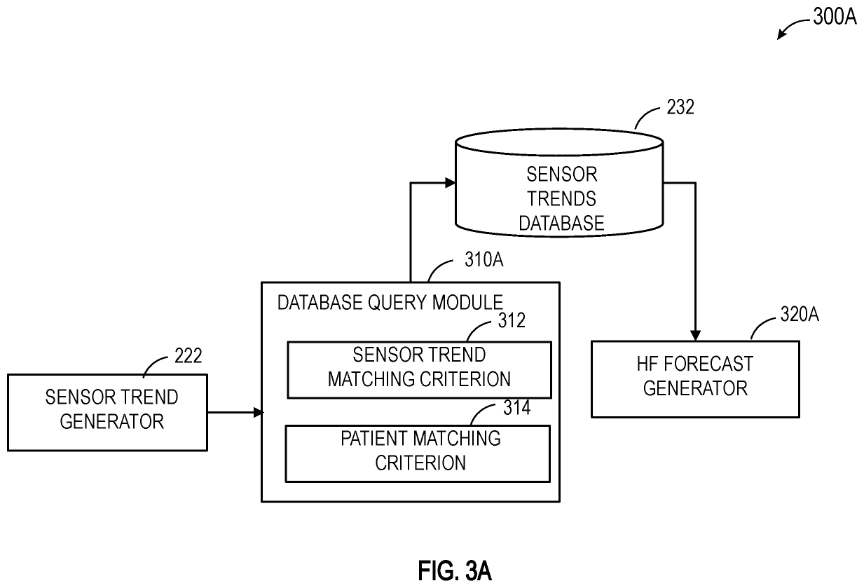

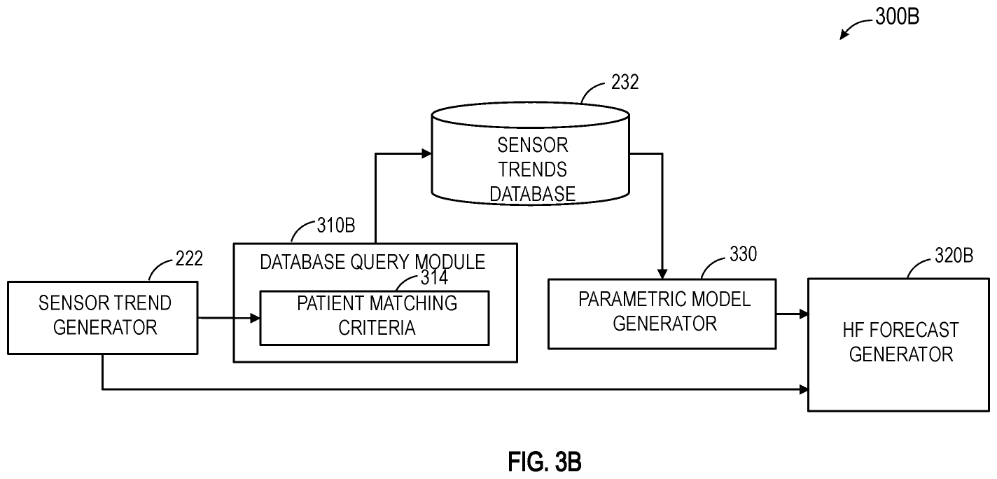

The heart failure forecast generator 228 may generate, for the patient, a projected sensor trend over a forecast period of time in future beyond the prediction time using the subset of the sensor trends selected by the database query module 226. The projected sensor trend indicates a forecast of future HF status of the patient. Various predictive modeling techniques may be used to establish a relationship (i.e., a model) between predictor variables (the variables used to predict a future response, such as pre-onset sensor data trend) and response variables (the variables to be predicted, such as post-onset sensor data trend indicative of a forecast of a future HF status). In an example, the heart failure forecast generator 228 may generate the projected sensor trend using a non-parametric model, in which the number of model parameters may grow with the size of the training data. In another example, the heart failure forecast generator 228 may generate a parametric model, which has a model structure with a fixed number of model parameters. The relationship between the predictor variables and the response variables may be established using a set of training data, such as a subset of the sensor trends selected from the sensor trends database 232 as training data. Examples of the non-parametric model includes K-nearest neighbor, decision trees, or support vector machines, among others. Examples of the parametric models include linear regression models, nonlinear regression models (e.g., logistic regression, polynomial regression, stepwise regression, ridge regression, and lasso regression), linear support vector machines, naïve Bayes, or perceptron models, among others. In some examples, the heart failure forecast generator 228 may generate the projected sensor trend using a machine learning (ML) model, examples of which may include neural networks, decision trees, Bayesian models, regression models, clustering, ensemble algorithms, or deep learning models, among others. The trained model may predict values of response variables (e.g., post-onset sensor data trend) for a given instance of predictor variable (e.g., pre-onset sensor data trend). Examples of the database query module 226 to identify matching sensor trends, and examples of the heart failure forecast generator 228 to generate a projected sensor trend, are discussed below with reference to FIGS. 3A-3B.

In some examples, the heart failure forecast generator 228 may generate additional statistical metrics using at least a portion of the selected subset of the stored sensor trends following the prediction time (e.g., 7 days, 14 days, or 30 days after the prediction time). Examples of such statistical metrics may include an upper bound of the projected sensor trend, a lower bound of the projected sensor trend, or a confidence interval about the projected sensor trend. The upper bound may be determined as the projected sensor trend (e.g., a central tendency of the selected subset of the stored sensor trends) plus a margin for each day during the forecast period. The lower bound may be determined as the projected sensor trend (e.g., a central tendency of the selected subset of the stored sensor trends) minus a margin for each day during the forecast period. The margin for the upper bound and the margin for the lower bound for a particular day may be determined based on variability of the sensor data (or sensor metric values or composite metric values) of the selected subset of the stored sensor trends on that particular day. In an example, the margin for the upper bound and the margin for the lower bound may both be twice the standard deviation of the sensor data (or sensor metric values or composite metric values) of the selected subset. In an examples, the upper bound and the lower bound may be determined using respective different margins.

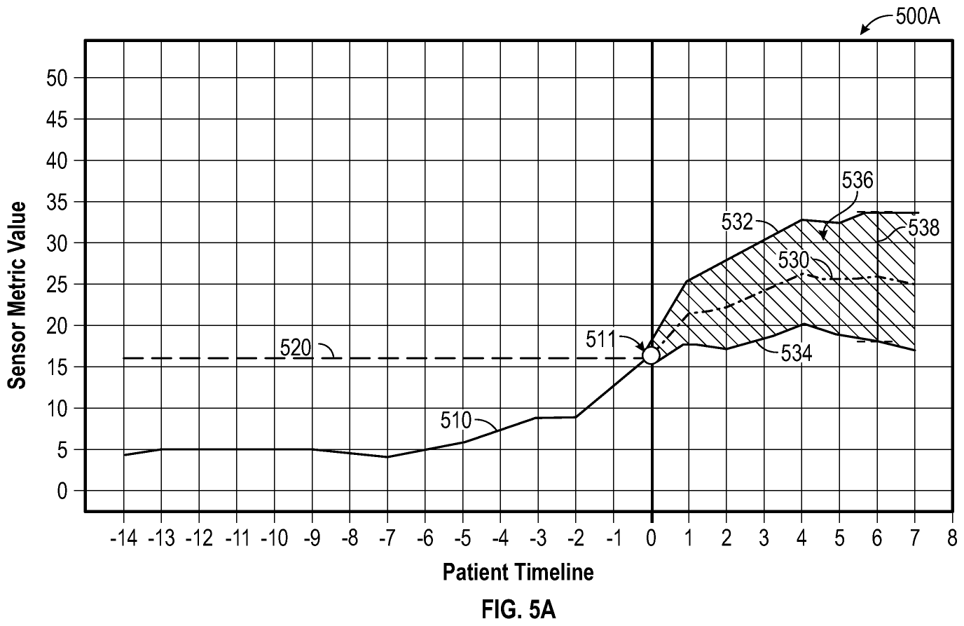

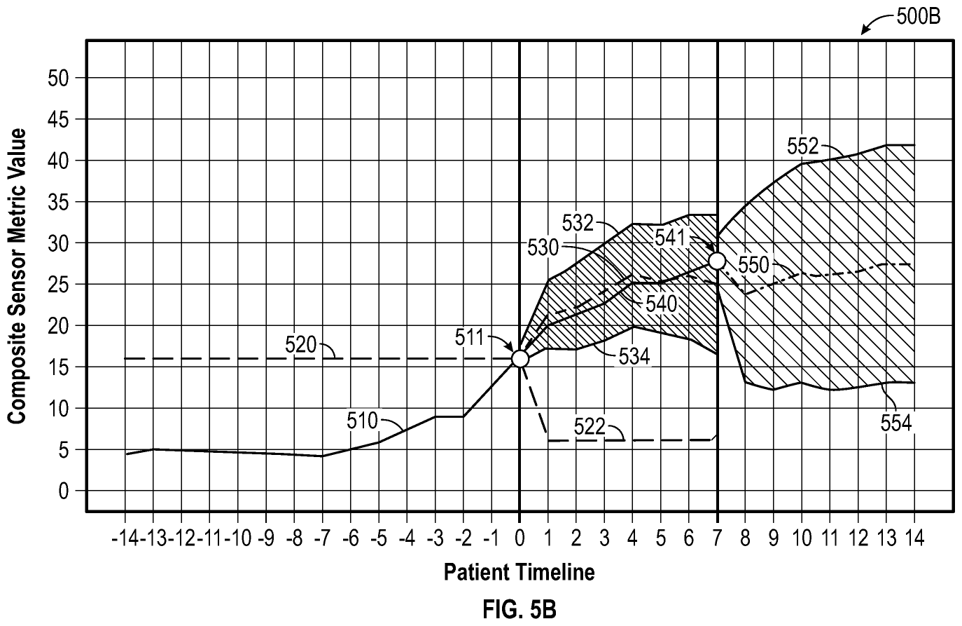

The projected sensor trend, optionally along with other statistical metrics such as the upper bound, the lower bound, and the confidence interval about the projected sensor trend, may be presented to a user via the user interface 240. The user interface 240, which may be implemented in the external system 125, may include a display for displaying a trajectory of the projected sensor trend over the time period beyond the specific prediction time, optionally with other information. In some examples, one or more of the upper bound, the lower bound, or the confidence interval about the projected sensor trend may be graphically or textually displayed along with the trajectory of the projected sensor trend. The lower and upper bounds may be represented by respective trajectories similar to the trajectory of the projected sensor trend, and the confidence interval about the projected sensor trend may be represented by a confidence interval band. Examples of the trajectory of the projected sensor trend and other statistical metrics such as upper and lower bounds and confidence interval about the projected sensor trend are discussed below, such as with reference to FIGS. 5A-5B.

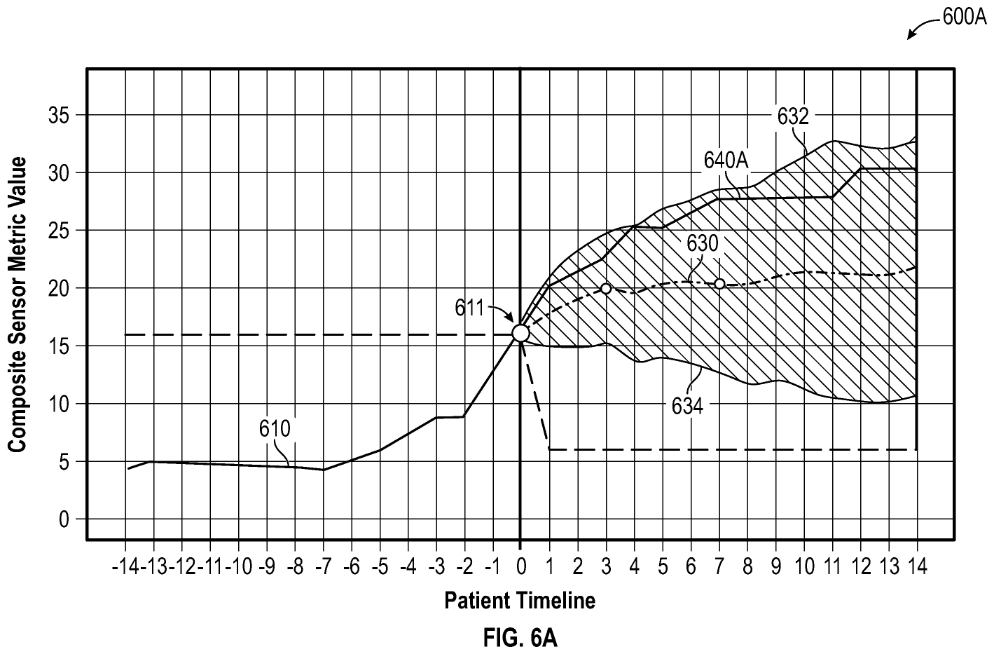

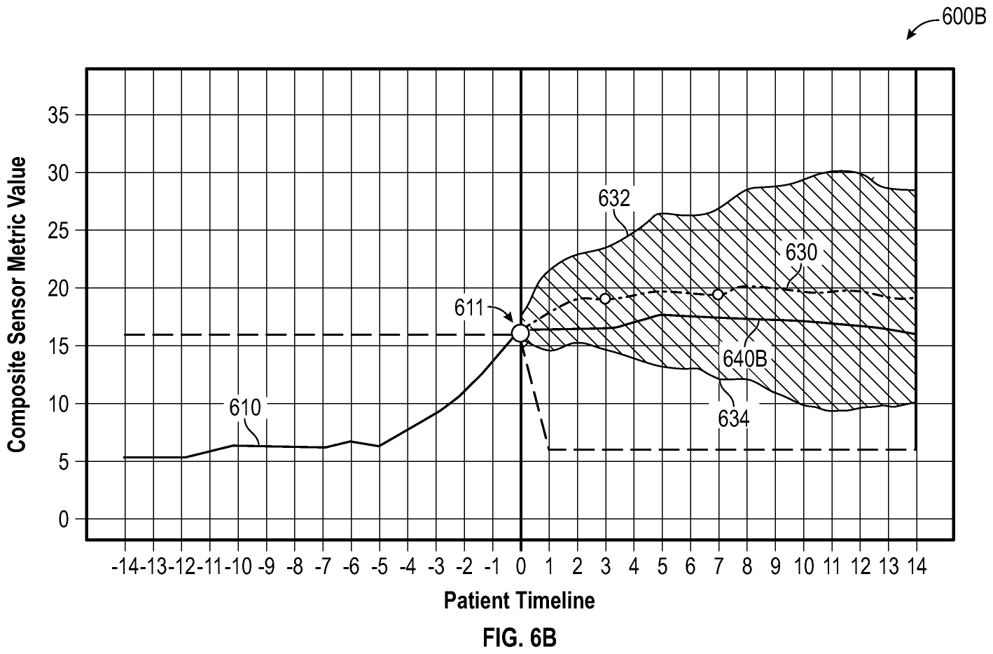

In some examples, the HF forecast generator 228 may generate a HF forecast report. The HF forecast report may be displayed on the display of the user interface 240. The report can include a textual or graphical comparison between the measured sensor data (or sensor metric values) and projected sensor data (or sensor metric values) on a particular day or over several days in the forecast period. For example, the report may indicate how good or bad a current sensor-indicated HF status actually is (based on the measured sensor data) relative to a previous forecast of the HF status, such as “current sensor-indicated HF status at 7-th day after the WHF onset is better than 75% of cases predicted based on historical data.” Based on such a comparison, a user (e.g., a clinician) may adjust HF management strategy, such as to titrate patient medication or device therapies. Examples of a comparison between the measured sensor trends and the projected sensor trends are discussed below, such as with reference to FIGS. 6A-6B.

Information displayed or otherwise presented to the user via the user interface 240 may also include one or more of the sensor data collected from the patient, monitored sensor trend of the patient, the subset of the stored sensor trends selected by the query module 226, among other intermediate measurements or computations. In some example, information of those patients associated with the selected subset of the sensor trends, including patient demographics, medical history such as medication, treatment, or hospital readmission, may also be displayed on the user interface 240. For example, re-admission statistics, such as a distribution of all past events that were followed by a 30-day readmission, may be determined for those patients associated with the selected subset of the sensor trends. Such a report of re-admission statistics of “similar patients” may be display to a user. A re-admission risk may be determined for the patient. The information may be presented in a table, a chart, a diagram, or any other types of textual, tabular, or graphical presentation formats. The presentation of the output information may include audio or other media format.

In some examples, alerts, alarms, emergency calls, or other forms of warnings may be generated to signal the system user (e.g., a clinician) about a forecast of an aggravated heart failure event in a forecast period, such as next 7 days, 14 days, or 30 days from the prediction time. In an example, alerts of distinct levels indicating high, medium, or low WHF risks may be generated based on one or more of the projected sensor trend, the upper bound, the lower bound, or the confidence interval about the projected sensor trend. In an example, a high alert may be generated if the projected sensor trend exceeds a threshold within a specific time period (e.g., 5 days) post the WHF onset, optionally along with a confidence interval within the specific time period falling below a threshold. In another example, a high alert may be generated if a rate of increase of the projected sensor trend exceeds a threshold. The alerts of distinct levels may be displayed on the user interface 240, and represented by different colors or identifiers.

The user interface 240 may include an input device that allows a user, such as the patient or a clinician, to provide input and other commands (e.g., setting parameter values or other programming operations). Examples of the input device may include a keyboard, an on-screen keyboard, a mouse, a trackball, a touchpad, a touch-screen, or other pointing or navigating devices. The user may use the input device to control one or more system functionalities such as sensing physiologic data via the sensor circuit 210, generating sensor trends via the sensor trend generator 222, detecting a heart failure event such as a WHF onset via the WHF detector 224, querying the database 232 via the database query module 226, or generating a projected sensor trend via the HF forecast generator 228, among others.

The optional therapy circuit 250 may be configured to deliver a therapy to the patient based on the forecast of future HF status, such as in response to the projected sensor trend satisfying a specific condition. Examples of the therapy may include electrostimulation therapy delivered to the heart, a nerve tissue, other target tissues, a cardioversion therapy, a defibrillation therapy, or drug therapy including delivering drug to a tissue or organ. In some examples, the therapy circuit 250 may modify an existing therapy, such as adjust a stimulation parameter or drug dosage, based on the forecast of future HF status. For example, drug dosage may be increased, or more aggressive cardiac pacing therapies may be delivered, in response to a forecast of a sustained worsening of HF status.

Although the discussion herein focuses on WHF event detection, this is meant only by way of example but not limitation. Systems, devices, and methods discussed in this document may also be suitable for detecting various sorts of diseases or for assessing risk of developing other worsened conditions, such as cardiac arrhythmias, heart failure decompensation, pulmonary edema, pulmonary condition exacerbation, asthma and pneumonia, myocardial infarction, dilated cardiomyopathy, ischemic cardiomyopathy, valvular disease, renal disease, chronic obstructive pulmonary disease, peripheral vascular disease, cerebrovascular disease, hepatic disease, diabetes, anemia, or depression, among others.

FIGS. 3A-3B illustrate generally examples of a portion of the heart failure monitor and forecast system 200 that may query the sensor trends database 232 for a subset of the sensor trends, and use the same to forecast the HF status of the patient. FIG. 3A illustrates a system portion 300A that may generate a projected sensor trend using a non-parametric approach. The system portion 300A includes a database query module 310A and a HF forecast generator 320A, which are embodiment of the database query module 226 and the HF forecast generator 228 of the system 200, respectively. The database query module 310A may compute a similarity score S(X, Yₖ) between the monitored sensor trend (X) of the patient and a stored senor trend Yₖ. The monitored sensor trend X may include sensor data of a particular sensor, a sensor metric trend, or a composite sensor trend. As discussed above, the monitored sensor trend X includes trended data up to the specific prediction time (day 0), such as a WHF onset or other HF forecast trigger events, or a user specified time such as a periodic forecast time. The stored senor trend Yₖ may correspond to a historical heart failure alert, and may include sensor data of a particular sensor, a sensor metric trend, or a composite sensor trend, such as one of the sensor metric trends 432A-432M as described above with reference to FIG. 4. The database query module 310A may compute the similarity score S(X, Yₖ) between a portion of the monitored sensor trend (X) with respect to the prediction time (e.g., WHF onset) and a portion of the stored senor trend Yₖ with respect to a prediction time (e.g., WHF onset). In an example, the similarity score S(X, Yₖ) may be computed using a 14-day monitored sensor trend (X) up to the device-detected WHF onset and a portion of the stored sensor trend Yₖ 14 days prior and up to the date of WHF onset.

The similarity score S(X, Yₖ) may include a distance metric or a correlation metric (e.g., a correlation coefficient) between the portion of the monitored sensor trend X and the portion of the stored sensor trend Yₖ. In an example, the monitored sensor trend X is a 14-day daily composite sensor trend X̅, and the stored sensor trend Yₖ is a 14-day daily composite sensor trend Y̅ₖ̅. The database query module 226 computes the similarity score S(X̅, Y̅ₖ̅) using a correlation coefficient between the sensor trends X̅ and Y̅ₖ̅.

The similarity score S(X, Yₖ) may be compared against a sensor trend matching criterion 312. The stored sensor trend Yₖ is deemed to match the monitored sensor trend X if the similarity score S(X, Yₖ) satisfies the sensor trend matching criterion 312, such as exceeding a threshold. In the event that the prediction time is a WHF onset, the matching sensor trend Yₖ thus identified has a pre-onset portion that follows a similar trend to the pre-onset portion of the monitored sensor trend X.

The database query module 310A may identify and select, from the stored sensor trends, all the matching sensor trends with corresponding similarity scores exceeding a threshold. In some examples, the database query module 310A may select a subset fewer than all of the matching sensor trends, such as those with top K similarity scores. In an example, a subset of approximately 20-25 matching sensor trends may be selected from the sensor trends database 232.

As an alternative to the composite sensor trends, in an example, the stored sensor trend Yₖ may comprise one or more individual sensor metric trends, such as N sensor metrics Yₖ(1), Yₖ(2), . . . , Yₖ(N) as illustrated in FIG. 4. Similarly, the monitored sensor trend X may also comprise one or more individual sensor metric trends, such as N sensor metrics X(1), X(2), . . . , X(N) corresponding to the sensor metrics Yₖ(1), Yₖ(2), . . . , Yₖ(N). The sensor metric X(i) and the corresponding sensor metric Yₖ(i) may be of the same type of sensor metric based on the same type of sensor. The database query module 310A may compute partial similarity scores for each of N sensor metrics, such as S(X(i), Yₖ(i)) between values of the monitored sensor metric trend X(i) and values of the stored sensor metric trend Yₖ(i). The partial similarity score may be a distance metric or a correlation metric (e.g., a correlation coefficient). The database query module 310A may compute the similarity score S(X, Yₖ) using a combination of the partial similarity scores, such as a weighted sum across all the sensor metrics 1 through N, each scaled by a corresponding weight factor a(i):

S(X,Yₖ)=Σᵢ₌₁^{N}a(i)*s(X(i),Yₖ(i)) (1)

In some examples, instead of accumulating over all N sensors metrics, a subset of the sensor metrics may be identified, and the partial similarity scores between X and Yₖ for only the identified subset of the sensor metrics may be combined to calculate the similarity score S(X, Yₖ). Such a subset of the sensor metrics may be identified using a comparison of the individual stored sensor metric trends, Yₖ(1) through Yₖ(N), to the composite sensor trend Y̅ₖ̅. Difference between the individual stored sensor metrics and the composite sensor trend Y̅ₖ̅ may be computed, such as based on an accumulated difference for 14 days prior and up to the WHF onset. The sensor metrics with the corresponding accumulated differences less than a threshold are deemed as following the trend of the composite sensor trend Yₖ and capturing significant HF worsening up to the WHF onset. The database query module 310A may then compute the similarity score S(X, Yₖ) using only the identified subset of the sensor metrics, such as a weighted combination similar to Equation (1) above.

In some examples, the database query module 310A may additionally use a patient matching criterion 314 to identify a subset of matching patients each having one or more of demographic information or medical history (e.g., prior heart failure events) matching that of the patient being analyzed, and to select stored sensor trends associated with the identified matching patients for HF status forecast. As described above, the stored sensor trends, such as the sensor metric trends 432A, 432B, . . . , 432M as shown in FIG. 4, may be generated using data collected from a plurality of patients. The sensor metric trends 432A, 432B, . . . , 432M may each be tagged with a patient identifier identifying the source of the data. For example, the sensor metric trends 432A and 432B are from patient 1, 432C is from patient 2, 432D-432F are from patient 3, etc. Patients with substantially identical demographic information or medical history may be identified as matching patients to the patient being analyzed. The database query module 310A may select the subset of the stored sensor trends only from those matching patients.

Including the patient demographics information or medical history in the process of selecting stored sensor trends, such as according to various examples as described herein, may help identify historical sensor trends that are more relevant to the HF event of the patient being evaluated, and improve the accuracy and robustness of HF status forecast for the patient.

The heart failure forecast generator 320A may generate, for the patient, a projected sensor trend over a forecast period of time in future beyond the prediction time using the subset of the sensor trends selected by the database query module 310A. The prediction time may be a HF event such as a WHF onset or a WHF termination, a user-specific time, or a time of periodic forecast. The forecast period may be, for example, 7 days, 14 days, or 30 days after the prediction time. The projected sensor trend indicates a forecast of future HF status of the patient. In an example, the projected sensor trend may be generated using a central tendency of at least a portion of the selected subset of the stored sensor trends during the forecast period. Examples of the central tendency includes a mean, a median, or a mode, among others. In some examples, additional statistical metrics may be generated using the selected subset of the stored sensor trends, such as the upper bound, lower bound, and confidence interval about the projected sensor trend, as discussed above with reference to FIG. 2.

FIG. 3B illustrates a system portion 300B, of the system 200, that may generate a projected sensor trend using a parametric approach. The system portion 300B includes a database query module 310B and a HF forecast generator 320B, which are embodiment of the database query module 226 and the HF forecast generator 228 of the system 200, respectively. The database query module 310B may use a patient matching criterion 314 to identify a subset of matching patients each having one or more of demographic information or medical history substantially identical to those of the patient being analyzed.

The system portion 300B includes a parametric model generator 330 configured to generate a parametric model using the sensor trends stored in the sensor trends database 232. In an example, the parametric model may be generated using the sensor trends from the matching patients identified by the database query module 310B. In an example, the parametric model may be a regression model being trained to model a relationship between (1) respective first portions of the matching patients' stored sensor trends, such as the data portions prior and up to respective prediction times (e.g., pre-WHF onset, such as sensor signal metric trend or composite sensor trend from day −14 to day 0 as shown in FIG. 4) and (2) respective second portions of the matching patients' stored sensor trends, such as the data portions after the respective prediction times (e.g., post-WHF onset, such as sensor signal metric trend or composite sensor trend from day 1 to day 14 as shown in FIG. 4). In an example, the regression model is a linear regression model. In another example, the regression model is a nonlinear regression model such as a logistic regression, a polynomial regression, a stepwise regression, a ridge regression, or a lasso regression. In an example, the regression model is an autoregressive (AR) model. The AR model is a linear regression of the data in the current series (e.g., sensor trend data at current or future times) against only the past values of the same series (e.g., sensor trend data at past times). In an example, an AR model with an order of p (also referred to as an AR(p) model) may model the value of a sensor trend Yₖ at time T as a linear combination of past p values of the sensor trend Yₖ at times T−1, T−2, . . . , T−p:

\tilde{Y}ₖ(T)=c+Σᵢ₌₁^{p}wᵢYₖ(T−i) (2)

where \tilde{Y}ₖ(T) is the predicted value of Yₖ at time T, w's are weight factors, and c is a constant. The AR(p) model may be trained to determine the model parameters (e.g., c and w's) using the data portions of the matching patients' stored sensor trends prior to the respective prediction times (e.g., pre-WHF onsets) and the data portions of the matching patients' stored sensor trends after the respective prediction times (e.g., post-WHF onsets).

The AR(p) model given in Equation (2) models Yₖ(T) uses p sensor trend values immediately before time T, that is, Yₖ(T−1) through Yₖ(T−p). In some examples, the p prior sensor trend values, Yₖ(T−1) through Yₖ(T−p), may be used to model a later sensor trend value beyond time T, e.g., at T+n:

\tilde{Y}ₖ(T+n)=c+Σᵢ₌₁^{p}wᵢYₖ(T−i),n≥1 (3)

The heart failure forecast generator 320B may generate, for the patient, a projected sensor trend over a forecast period of time in future beyond the prediction time using the monitored sensor trend of the patient and a trained parametric model, such as a trained regression model. As discussed above, the prediction time may be a HF event such as a WHF onset or a WHF termination, a user-specific time, or a time of periodic forecast. The forecast period may be, for example, 7 days, 14 days, or 30 days after the prediction time. In an example, the heart failure forecast generator 320B may apply the monitored sensor trend from day −14 to day 0 (the prediction time), X(−14) to X(0), to the trained AR(p) model as given in Equations (2) or (3) (with p=15), to determine the predicted sensor trend. For example, the predicted value at a particular date (q), X(q), during the forecast period (Q) may be calculated as follows:

\tilde{X}(q)=c+Σᵢ₌₁¹⁵wᵢX(1−i), for q=1,2, . . . ,Q (4)

The Equation (4) above is used to calculate the predicted value at any date during the forecast period Q using the monitored sensor trend from day −14 to day 0, i.e., X(−14) to X(0). Alternatively, the predicted value at a later date may be determined using a previously predicted value at an earlier date, in accordance with Equation (2). For example, to use an AR(p) model of Equation (2) with a model order p=15, the predicted sensor trend value at day 8 after the WHF onset, \tilde{X}(8), may be estimated using the previously estimated sensor trend values \tilde{X}(1) through \tilde{X}(7) for day 1 to day 7, and the monitored sensor trend data for 7 days prior and up to the date of WHF onset, X(−7) through X(0).

The projected sensor trend indicates a forecast of future HF status of the patient for a specified forecast period post the prediction time. In some examples, additional statistical metrics may be generated using the selected subset of the stored sensor trends, such as the upper bound, lower bound, and confidence interval about the projected sensor trend, as discussed above with reference to FIG. 2.

FIGS. 5A-5B are diagrams illustrating exemplary trajectories of the projected sensor trend indicating a forecast of HF status, which may be displayed on a display of the user interface 240. Other statistical metrics, such as trajectories of the upper and lower bounds of the projected sensor trends and the confidence interval band, are also shown. FIG. 5A illustrates an event-triggered HF status forecast diagram 500A. A composite sensor trend 510, such as generated by the sensor trend generator 222 that trends a composite signal metric over time, may be compared to a WHF onset threshold 520. The WHF detector 224 detects a WHF onset 511 when the composite sensor trend 510 crosses above the WHF onset threshold 520. The time of the WHF onset is referred to as day 0 on the x-axis. An alert of WHF onset 511 may be generated. The detection of the WHF onset triggers HF forecast. The database query module 226 may take a portion of the composite trend 510 corresponding to 14 days prior and up to the WHF onset 511, and query the sensor trends database 232 for one or more sensor trends stored therein each having respective first data portions (e.g., 14 days prior and up to respective WHF onsets) that match the data portion taken from the composite trend 510. As discussed above with reference to FIG. 2, a match may be based on a similarity metric, such as a correlation coefficient. A projected sensor trend 530 may be generated using a median (or other central tendency measures) of respective second data portions of the selected matching sensor trends following the WHF onset 511. The length of the second data portions, or the length of forecast, may be specified by a user, and depends on the data length of the stored sensor trends. By way of example and not limitation, a 7-day forecast post the WHF onset is shown in FIG. 5. An upper bound 532 and a lower bound 534 may also be determined based on variability (e.g., standard deviation) of the second data portions of the selected matching sensor trends following the WHF onset 511. The region between the upper bound 532 and a lower bound 534 defines a confidence interval band 536 about the projected sensor trend 530. The confidence interval may be determined for a particular post-onset time, such as the confidence interval 538 at day 6 (i.e., 6 days after the WHF onset 511).

FIG. 5B illustrates a periodic HF status forecast diagram 500B. Similar to the event-triggered forecast as shown in FIG. 5A, a 7-day HF forecast may be performed in response to a detection of WHF onset 511 at day 0, and a projected sensor trend 530, along with the upper and lower bounds 532 and 534 thereof, may be displayed. Upon the detection of WHF onset 511, the HF detection threshold may be updated from the onset threshold 520 to a reset threshold that may be used for detecting HF termination. Sensor data may be monitored and trended from day 0 to day 7. The monitored sensor trend 540 may be displayed with the previously projected sensor trend 530. In the example as shown, the monitored sensor trend 540 closely follows the projected sensor trend 530, indicating a high forecast accuracy. Periodic forecast may be performed every 7 days. At day 7, the monitored sensor trend 540 stays above the reset threshold 522, suggesting that the WHF is unterminated. A re-alert may be generated at 541. A scheduled periodic forecast may be imitated one the day of re-alert 541, and another projected sensor trend 550 for the next 7 days post re-alert may be generated. Upper bound 552 and lower bound 554 of the projected sensor trend may be generated by the database query module 226 and the HF forecast generator 228 through a similar process of generating the projected sensor trend 530 and the upper and lower bounds 532 and 534, as discussed above.

FIGS. 6A-6B are diagrams illustrating examples of comparing and contrasting a projected composite sensor trend with a measured composite sensor trend during the same forecast period. The forecast is triggered by the detection of a WHF onset event 611, similar to that illustrated in FIGS. 5A-5B. The forecast period extends to 14 days after the WHF onset 611 (day 0 to day 14). Such a comparison, when presented graphically or textually to a user, may assist the clinician to make clinical decisions of initiating or adjusting HF management strategies, such as to titrate medication or device therapies. FIG. 6A illustrates a HF status forecast diagram 600A representing a 14-day forecast post WHF onset 611. In this example, despite a forecast of a sustained elevation of the composite metric trend (indicating a steady deterioration of HF status) for the next 14 days, as indicated by the projected sensor trend 630, no treatment was administered, or no change to the existing treatment regimen was made, at the time of WHF onset 611. As a result, the actual monitored composite sensor trend 640A shows a steady increase and approaches the upper bound 632 of the 14-day the projected sensor trend 630, indicating an aggravated HF status.

FIG. 6B illustrates a HF status forecast diagram 600B representing a 14-day forecast post WHF onset 611. In this example, in response to a forecast of a 14-day sustained elevation of the composite metric trend as indicated by the projected sensor trend 630, a treatment was administered, or an existing treatment regimen was adjusted based on the forecast of HF status, at the time of WHF onset 611. As a result, the monitored composite sensor trend 640A shows a substantially flat trend staying below the projected sensor trend 630 for the entire 14-day forecast period. With the early treatment assisted by the HF forecast such as in accordance with various examples described herein, the patient's HF status is better controlled, and further exacerbation of HF status may be prevented or lessened.

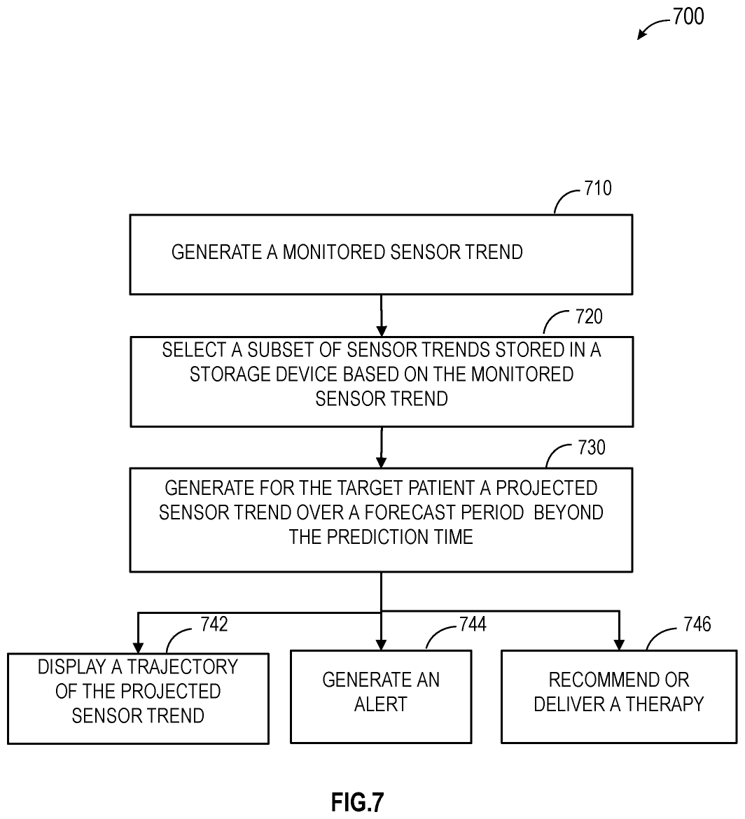

FIG. 7 illustrates generally an example of a method 700 for monitoring heart failure (HF) in a patient. The method 700 may be implemented in and executed by ambulatory medical device, such as an implantable or wearable medical device, or a remote patient management system. In various examples, the method 700 may be implemented in and executed by the AMD 110, one or more devices in the external system 125, or the heart failure monitor and forecast system 200 or a modification thereof.

The method 700 commences at step 710, where a sensor trend may be generated using sensor data collected from a patient such as by the sensor circuit 210. Examples of the sensor data may include ECG, EGM, heart rate signal, physical activity signal, or posture signal, a thoracic or cardiac impedance signal, arterial pressure signal, pulmonary artery pressure signal, left atrial pressure signal, RV pressure signal, LV coronary pressure signal, coronary blood temperature signal, blood oxygen saturation signal, heart sound signal, physiologic response to activity, apnea hypopnea index, one or more respiration signals such as a respiratory rate signal or a tidal volume signal, brain natriuretic peptide, blood panel, sodium and potassium levels, glucose level and other biomarkers and bio-chemical markers, among others. The sensors used for collecting implantable, wearable, or otherwise ambulatory sensor or electrodes associated with the patient.

The sensor trend for the patient, also referred to as a monitored sensor trend, may be generated using the sensor data collected up to a prediction time. The prediction time is the time when a HF status forecast is initiated. In an example, the HF status forecast may be triggered by a forecast trigger event. Examples of the forecast trigger event may include a WHF onset event or a WHF termination event, which may be detected by the WHF detector 224 based on changes of the monitored sensor trend over time, as discussed above with reference to FIG. 2. Other examples of the forecast trigger event may include medical events such as admission to a hospital, discharge from a hospital, development of particular heart failure symptom or comorbidity, among others. In some examples, the HF status forecast may be initiated by a user command, or that the prediction time may be specified by a user, or according to a specific schedule. In some examples, the HF status forecast may be performed periodically. For example, the forecast may be performed nightly, weekly, or monthly.

In an example, the monitored sensor trend may be a time-series of sensor signal metric values. The sensor signal metric may include a statistical or morphological feature extracted from the sensor signal. In some examples, the monitored sensor trend may be a composite sensor trend generated using a fusion of sensor data collected from two or more different sensors, a fusion of two or more different sensor metrics derived from the same sensor, or a fusion of two or more different sensors metrics respectively derived from different sensors. Various fusion algorithms may be used. In an example, the fusion includes a weighted combination of data from different sensors or values of different sensor metrics. By way of example and not limitation, a monitored sensor trend may include trended sensor data, sensor metric values, or composite signa metric values, for 14 days prior and up to the date of WHF onset (that is, day −14 to day 0), as illustrated in FIGS. 5A-5B and 6A-6B.

At 720, information about patients' data collected from a plurality of patients may be received. The patients' data may be stored in a storage device, such as the storage device 230. In an example, the stored patients' data include sensor trends that are generated using the patients' historical sensor data. In an example, at least a subset of sensor trends may be selected from a sensor trends database stored in the storage device. The sensor trends in the database may be generated using sensor data collected from a plurality of patients, and include trends of sensor data, trends of sensor signal metrics, or composite sensor trends each representing a trend of a composite signal metric as a combination of two or more sensor signal metrics. An example of the sensor trends database is shown in FIG. 4, where N sensor signal metrics and a composite sensor signal metric are each trended over a period extending from 14 days prior to the WHF onset to 14 days after the date of WHF termination.

The subset of sensor trends may be selected from a sensor trends database based on the monitored sensor trend of the patient obtained from step 710. In an example, the sensor trends database may be queried (such as using the database query module 226 of the system 200) for a subset of the sensor trends that satisfy specific search criteria in accordance with the monitored sensor trend of the patient. The search criteria may include on or more of a sensor trend matching criterion, or a patient matching criterion, as discussed above with reference to FIGS. 3A-3B. The sensor trend matching criterion may involve computing similarity scores between the monitored sensor trend of the patient up to the prediction time and at least a first portion of the stored sensor trends (the portion prior and up to the prediction time), and identifying the matching sensor trends as those having the corresponding similarity scores exceeding a threshold. The patient matching criterion may involve identifying a subset of matching patients each having one or more of demographic information or medical history (e.g., prior heart failure events) matching that of the patient being analyzed.

At 730, a projected sensor trend may be generated for the patient using the selected subset of the stored sensor trends, such as via the heart failure forecast generator 228. The forecast period may be, for example, 7 days, 14 days, or 30 days after the prediction time. The projected sensor trend, which indicates a forecast of future HF status over a specific forecast period of time in future beyond the prediction time, may be generated using a predictive model. In one example, the prediction model may be a non-parametric model, such as one of a K-nearest neighbor model, a decision tree, or a support vector machine model, among others. In another example, the prediction model may be a parametric model, such as one of a linear regression model, a nonlinear regression model, or a linear support vector machine, a naïve Bayes model, or a perceptron model, among others. A non-parametric approach for generating the projected sensor trends is discussed above with reference to FIG. 3A. As discussed therein, in an example, the projected sensor trend may be generated using a central tendency of at least a portion of the selected subset of the stored sensor trends during a forecast period following the prediction time. A parametric approach for generating the projected sensor trends is discussed above with reference to FIG. 3B. As discussed therein, a parametric model (such as a regression model) may be trained using the selected sensor trends such as obtained from step 720. The parameter model is trained to establish a relationship, as characterized by model parameters, between predictor variables (e.g., sensor data trend prior to the prediction time) and response variables e.g., sensor data after the prediction time). Then, the monitored sensor trend up to the prediction time (such as 14-day signal trend prior and up to the date of WHF onset) may be applied to the trained parametric model to generate the projected sensor trend.

In some examples, in addition to the projected sensor trend, other statistical metrics may be generated using the selected subset of the stored sensor, such as the upper bound, lower bound, and confidence interval about the projected sensor trend, as discussed above with reference to FIG. 2.

The projected sensor trend, optionally along with other statistical metrics such as the upper bound, the lower bound, and the confidence interval about the projected sensor trend, may be presented to a user or a process. At 742, a trajectory of the projected sensor trend may be displayed on a display unit. The trajectory may extend for a forecast period of time in future beyond the prediction time, such as 7 days or 14 days. One or more of the upper bound, the lower bound, or the confidence interval about the projected sensor trend may be graphically or textually displayed along with the trajectory of the projected sensor trend, as discussed above with reference to FIGS. 5A-5B and 6A-6B.

Additionally or alternatively, at 744, an alert may be generated to signal the system user (e.g., a clinician) about a forecast of an aggravated heart failure event. In some examples, alerts of distinct levels indicating high, medium, or low WHF risks may be generated based on one or more of the projected sensor trend, the upper bound, the lower bound, or the confidence interval about the projected sensor trend. Additionally or alternatively, at 746, a therapy may be delivered to the patient, or an existing therapy may be adjusted, based on the forecast of future HF status, such as in response to the projected sensor trend satisfying a specific condition.

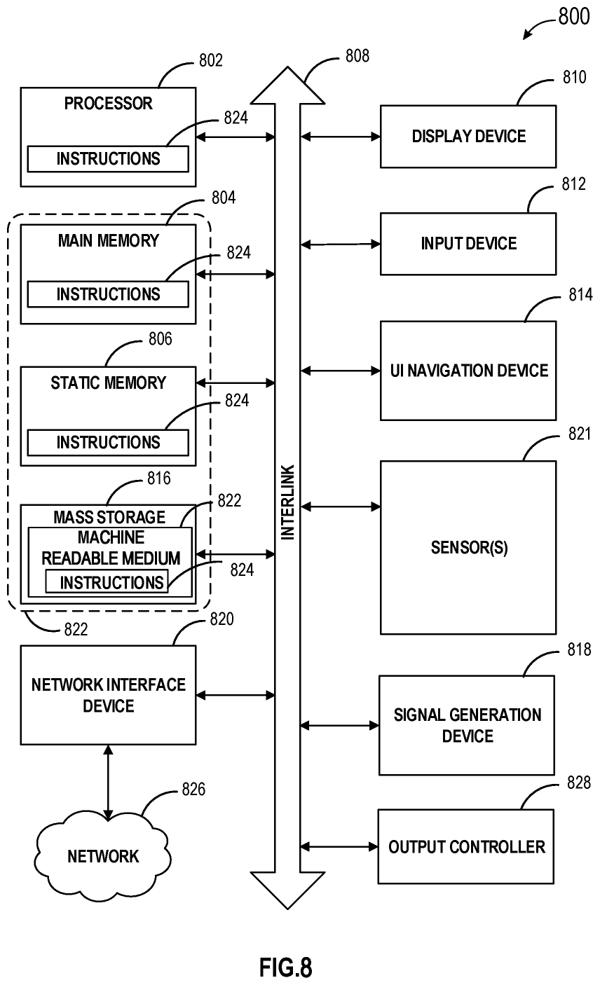

FIG. 8 illustrates generally a block diagram of an example machine 800 upon which any one or more of the techniques (e.g., methodologies) discussed herein may perform. Portions of this description may apply to the computing framework of various portions of the IMD, or the external programmer.

In alternative embodiments, the machine 800 may operate as a standalone device or may be connected (e.g., networked) to other machines. In a networked deployment, the machine 800 may operate in the capacity of a server machine, a client machine, or both in server-client network environments. In an example, the machine 800 may act as a peer machine in peer-to-peer (P2P) (or other distributed) network environment. The machine 800 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specific operations when operating. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 800 may include a hardware processor 802 (e.g., a central processing unit (CPU), a graphics processing unit (GPU), a hardware processor core, or any combination thereof), a main memory 804 and a static memory 806, some or all of which may communicate with each other via an interlink (e.g., bus) 808. The machine 800 may further include a display unit 810 (e.g., a raster display, vector display, holographic display, etc.), an alphanumeric input device 812 (e.g., a keyboard), and a user interface (UI) navigation device 814 (e.g., a mouse). In an example, the display unit 810, input device 812 and UI navigation device 814 may be a touch screen display. The machine 800 may additionally include a storage device (e.g., drive unit) 816, a signal generation device 818 (e.g., a speaker), a network interface device 820, and one or more sensors 821, such as a global positioning system (GPS) sensor, compass, accelerometer, or other sensor. The machine 800 may include an output controller 828, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 816 may include a machine readable medium 822 on which is stored one or more sets of data structures or instructions 824 (e.g., software) embodying or utilized by any one or more of the techniques or functions described herein. The instructions 824 may also reside, completely or at least partially, within the main memory 804, within static memory 806, or within the hardware processor 802 during execution thereof by the machine 800. In an example, one or any combination of the hardware processor 802, the main memory 804, the static memory 806, or the storage device 816 may constitute machine-readable media.

While the machine-readable medium 822 is illustrated as a single medium, the term “machine readable medium” may include a single medium or multiple media (e.g., a centralized or distributed database, and/or associated caches and servers) configured to store the one or more instructions 824.

The term “machine readable medium” may include any medium that is capable of storing, encoding, or carrying instructions for execution by the machine 800 and that cause the machine 800 to perform any one or more of the techniques of the present disclosure, or that is capable of storing, encoding or carrying data structures used by or associated with such instructions. Non-limiting machine-readable medium examples may include solid-state memories, and optical and magnetic media. In an example, a massed machine-readable medium comprises a machine readable medium with a plurality of particles having invariant (e.g., rest) mass. Accordingly, massed machine-readable media are not transitory propagating signals. Specific examples of massed machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 824 may further be transmitted or received over a communications network 826 using a transmission medium via the network interface device 820 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as WiFi®, IEEE 802.16 family of standards known as WiMax®), IEEE 802.15.4 family of standards, peer-to-peer (P2P) networks, among others. In an example, the network interface device 820 may include one or more physical jacks (e.g., Ethernet, coaxial, or phone jacks) or one or more antennas to connect to the communications network 826. In an example, the network interface device 820 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 800, and includes digital or analog communications signals or other intangible medium to facilitate communication of such software.

Various embodiments are illustrated in the figures above. One or more features from one or more of these embodiments may be combined to form other embodiments.

The method examples described herein can be machine or computer-implemented at least in part. Some examples may include a computer-readable medium or machine-readable medium encoded with instructions operable to configure an electronic device or system to perform methods as described in the above examples. An implementation of such methods may include code, such as microcode, assembly language code, a higher-level language code, or the like. Such code may include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A system for monitoring heart failure (HF) in a patient, the system comprising:
a HF predictor circuit configured to:
generate a monitored sensor trend using sensor data collected up to a prediction time from the patient; and
generate a projected sensor trend for the patient over a forecast period of time in future beyond the prediction time using at least the monitored sensor trend of the patient, the projected sensor trend indicative of a forecast of future HF status of the patient.

2. The system of claim 1, comprising a storage device configured to store sensor trends data collected from a plurality of patients, the stored sensor trends each including a first portion prior to a prediction time and a subsequent second portion post the prediction time, and
wherein the HF predictor circuit is configured to:
based on the monitored sensor trend of the patient, select at least a subset of the stored sensor trends; and
generate the projected sensor trend for the patient further using the selected subset of the stored sensor trends.

3. The system of claim 2, wherein:
the monitored sensor trend includes a composite sensor trend based on sensor data from two or more sensors associated with the patient; and
the stored sensor trends include at least one composite sensor trend based on sensor data from two or more sensors associated with one of the plurality of patients.

4. The system of claim 2, wherein the HF predictor circuit is configured to:
determine similarity indices between (1) the monitored sensor trend of the patient up to the prediction time and (2) respective first portions of the stored sensor trends;
select at least the subset of the stored sensor trends based on the similarity indices; and
generate the projected sensor trend for the patient using a central tendency of respective second portions of the selected subset of the stored sensor trends.

5. The system of claim 4, wherein the HF predictor circuit is configured to determine the similarity indices using distance metrics or correlation metrics between the monitored sensor trend of the patient and the respective first portions of the stored sensor trends.

6. The system of claim 4, wherein the stored sensor trends include a composite sensor trend generated using sensor data from two or more sensors associated with one of the plurality of patients, and the HF predictor circuit is configured to:
identify, from the two or more sensors used for generating the composite sensor trend, one or more sensors with respective sensor trends that are substantially similar to the composite sensor trend;
determine similarity indices between (1) the monitored sensor trend of the patient and (2) respective first portions of the sensor trends corresponding to the identified one or more sensors;
select a subset of the sensor trends corresponding to the identified one or more sensors with respective similarity indices exceeding a threshold; and
for the patient, generate the projected sensor trend using a central tendency of respective second portions of the selected subset of the sensor trends corresponding to the identified one or more sensors.

7. The system of claim 2, wherein the HF predictor circuit is configured to:
generate a parametric predictive model representing a relationship between (1) respective first portions of the selected subset of the sensor trends and (2) respective second portions of the selected subset of the sensor trends; and
apply the monitored sensor trend to the parametric predictive model to generate the projected sensor trend for the patient.

8. The system of claim 7, wherein the parametric predictive model is a regression model.

9. The system of claim 2, wherein the HF predictor circuit is configured to:
identify, from the plurality of patients with sensor trends stored in the storage device, one or more matching patients with respective demographic or medical history information substantially similar to that of the patient; and
select the subset of the stored sensor trends from the one or more matching patients.

10. The system of claim 2, wherein the HF predictor circuit is configured to generate, using the selected subset of the stored sensor trends, at least one of an upper bound of the projected sensor trend, a lower bound of the projected sensor trend, or a confidence interval about the projected sensor trend.

11. The system of claim 1, wherein the prediction time includes at least one of: a time of detection of worsening heart failure (WHF) onset; a time of detection of WHF termination; or a periodic prediction time.

12. The system of claim 1, comprising a display configured to display a trajectory of the projected sensor trend over the forecast period.

13. A method for monitoring heart failure (HF) in a patient, the method comprising:
generating a monitored sensor trend using sensor data collected up to a prediction time from the patient; and
generating, for the patient, a projected sensor trend over a forecast period of time in future beyond the prediction time using at least the monitored sensor trend, the projected sensor trend indicative of a forecast of future HF status of the patient.

14. The method of claim 13, comprising:
receiving sensor trends data collected from a plurality of patients and stored in a storage device, the sensor trends each including a first portion prior to a prediction time and a subsequent second portion post the prediction time;
selecting at least a subset of the stored sensor trends based on the monitored sensor trend of the patient; and
generating the projected sensor trend for the patient further using the selected subset of the stored sensor trends.

15. The method of claim 14, wherein:
the monitored sensor trend includes a composite sensor trend based on sensor data from two or more sensors associated with the patient; and
the stored sensor trends include at least one composite sensor trend based on sensor data from two or more sensors associated with one of the plurality of patients.

16. The method of claim 14, wherein:
selecting at least the subset of the stored sensor trends is based on similarity indices between (1) the monitored sensor trend of the patient up to the prediction time and (2) respective first portions of the stored sensor trends; and
generating the projected sensor trend includes using a central tendency of respective second portions of the selected subset of the stored sensor trends.

17. The method of claim 14, comprising:
generating a parametric predictive model representing a relationship between (1) respective first portions of the selected subset of the sensor trends and (2) respective second portions of the selected subset of the sensor trends; and
applying the monitored sensor trend to the parametric predictive model to generate the projected sensor trend for the patient.

18. The method of claim 14, wherein selecting at least the subset of the stored sensor trends includes:
identifying, from the plurality of patients with sensor trends stored in the storage device, one or more matching patients with respective demographic or medical history information substantially similar to that of the patient; and
selecting the subset of the stored sensor trends from the one or more matching patients.

19. The method of claim 13, wherein the prediction time includes at least one of: a time of detection of worsening heart failure (WHF) onset; a time of detection of WHF termination; or a periodic prediction time.

20. The method of claim 13, comprising displaying a trajectory of the projected sensor trend over the forecast period.
